(19) United States
(12) Patent Application Publication
(10) Pub. No.: US 2004/0210248 A1
(43) Pub. Date: Oct. 21, 2004

(54) Apparatus, method and assembly for delivery of intra-bronchial devices

(75) Inventors: Lucas S. Gordon, Sammamish, WA (US); James M. Kutsko, Woodinville, WA (US); David H. Dillard, Redmond, WA (US)

Correspondence Address: KNOBBE MARTENS OLSON & BEAR LLP, 2040 MAIN STREET, FOURTEENTH FLOOR, IRVINE, CA, 92614, US

(73) Assignee: Spiration, Inc.

(21) Appl. No.: 10/387,963

(22) Filed: Mar. 12, 2003

(51) Int. Cl. A61M 29/00

(52) U.S. Cl. 606/191

(57) ABSTRACT

An assembly delivers an intra-bronchial device into an air passageway. The assembly includes a delivery catheter having a deployment lumen with a distal end arranged to receive the intra-bronchial device in a delivery configuration, and arranged to be passed down a trachea and advanced to a location for deployment of the intra-bronchial device, and to deploy the intra-bronchial device by retracting the deployment catheter. The assembly further includes a bronchial device supply tool associated with the distal end, and arranged to carry the intra-bronchial device in a storage configuration and to load the intra-bronchial device in a delivery configuration into the deployment lumen of distal end, and the intra-bronchial device carried in the supply tool in a storage configuration. An embodiment of the supply tool includes a storage portion, a delivery portion, and a configuration-changing portion coupled between the storage portion and the delivery portion.

### BACKGROUND

Advances in medicine are providing improved intra-bronchial devices for delivery to portions of the respiratory system, particularly the lungs and bronchi. The intra-bronchial devices typically are delivered into the respiratory system by deployment from a catheter placed into the respiratory system through a working channel of a bronchoscope.

Storage, transportation, and placing the devices in a delivery configuration present unique problems not previously addressed in the medical community. The devices are small, fragile, and difficult to handle and store. As a result, it may be difficult to physically select a desired device, load the device into a catheter, and deliver the device to the intended location in a respiratory system without a risk of compromising the device. Furthermore, the devices may require sterilization and isolation from contaminates before implantation, and may have features that interfere with delivery, such as anchors. Another factor is that several different sizes of devices must be available during a procedure because the diameter of the air passageways varies, continuously becoming smaller in the distal direction.

Intra-bronchial devices may be used for a variety of therapeutic objectives. For example, these objectives may include, but are not limited to, treatment of chronic obstructive pulmonary disease (COPD) by lung volume reduction as described in U.S. Pat. No. 6,258,100, METHOD OF REDUCING LUNG SIZE, and U.S. Pat. No. 6,293,951, LUNG REDUCTION DEVICE, SYSTEM, AND METHOD; and provision of a therapeutic agent as described in U.S. Pat. No. ______(application Ser. No. ______ [1759-33], filed Dec. 11, 2002), DEVICE AND METHOD FOR INTRA-BRONCHIAL PROVISION OF A THERAPEUTIC AGENT, all of which are commonly owned by the assignee of this document and which are incorporated herein by reference. Intra-bronchial devices are presently available with several different types of structures, including devices that preclude air flow in both directions, preclude air flow in one direction as described in the '951 patent, or do not preclude air flow at all as described in U.S. Pat. No. ______ (Application number ______ [1759-33-3).

Furthermore, intra-bronchial devices may include anchoring members or other features that may be advantageously served by appropriate storage. For example, devices with anchoring members are described in U.S. Pat. No. ______ (application Ser. No. 09/951,105, filed Sep. 11, 2001), REMOVABLE LUNG REDUCTION DEVICES, SYSTEMS, AND METHODS; U.S. Pat. No. ______ (application Ser. No. 10/104,487, filed Mar. 20, 2002), REMOVABLE ANCHORED LUNG VOLUME REDUCTION DEVICES AND METHODS; and U.S. Pat. No. ______ (application Ser. No. 10/150,547, filed May 17, 2002), REMOVABLE ANCHORED LUNG VOLUME REDUCTION DEVICES AND METHODS, hereafter collectively referred to as “the anchored device applications.” The anchored device applications are all commonly owned by the assignee of the present invention and are incorporated herein by reference. Obstructing members, anchoring members, and other features of intra-bronchial devices are typically collapsed or compressed into a delivery configuration to fit into a deployment channel, such as a catheter lumen, for delivery into an air passageway. However, the obstructing members, anchoring members, or other features may require storage in a configuration different than their delivery configuration to maintain functionality. For example, elasticity of an intra-bronchial device obstructing member or elasticity of anchoring members may be degraded by storage in a delivery configuration.

In addition, an intra-bronchial device may include a medicant or a therapeutic agent that requires special storage considerations, or that does not tolerate physical handling that might be required to place it in a delivery configuration or load it into a deployment lumen. For example, see U.S. Pat. No. ______ (application Ser. No. 10/178,073, filed Jun. 21, 2002), INTRA-BRONCHIAL DEVICE THAT PROVIDES A MEDICANT INTRA-BRONCHIALLY TO THE PATIENT; and U.S. Pat. No. ______[1759-33-3] ______ (application Ser. No. 10/______, filed Dec. 11, 2002), DEVICE AND METHOD FOR INTRA-BRONCHIAL PROVISION OF A THERAPEUTIC AGENT, all of which are commonly owned by the assignee of the present invention and which are incorporated herein by reference.

Also, treatment involving intra-bronchial devices requires a provider to have different sizes of intra-bronchial devices readily available during a procedure to match the different diameters of air passageways. This is because the air passageways into which the intra-bronchial devices may be deployed branch out, much like the roots of a tree wherein each branch is proportionally smaller. No single intra-bronchial device is presently able to fit an entire range of internal air passageway diameters; therefore, several different sizes of intra-bronchial devices should be available when treating a patient.

In view of the foregoing, there is a continuing need for an improved device, assembly, and method for storage, transportation, delivery, and implantation of intra-bronchial devices. The present invention is directed to such a device, assembly, and method.

### SUMMARY OF THE INVENTION

The invention provides an assembly for delivering an intra-bronchial device into an air passageway. The assembly includes a delivery catheter having a deployment lumen with a distal end arranged to receive the intra-bronchial device in a delivery configuration, and arranged to be passed down a trachea and advanced to a location in an air passageway for deployment of the intra-bronchial device, and to deploy the intra-bronchial device by retracting the deployment catheter. The assembly further includes a bronchial device supply tool associated with the distal end, and arranged to carry the intra-bronchial device in a storage configuration and to load the intra-bronchial device in a delivery configuration into the deployment lumen of distal end. The assembly still further provides the intra-bronchial device carried in the supply tool in a storage configuration. The delivery catheter may include a deployment catheter having a deployment lumen, and a stabilization member arranged to be carried in the deployment lumen and to stabilize the intra-bronchial device at the location during retraction of the distal end. The supply tool may include a storage portion, a delivery portion, and a configuration-changing portion coupled between the storage portion and the delivery portion.

Another embodiment of the invention provides an assembly for delivering an intra-bronchial device into an air passageway. The assembly includes a delivery catheter having a distal sheath arranged to receive the intra-bronchial device in a delivery configuration and to deploy the intra-bronchial device by unsheathing, the distal sheath being further arranged to be passed down a trachea and advanced to a location in an air passageway for deployment of the intra-bronchial device. The assembly also includes a bronchial device supply tool removably coupled to the distal end and arranged to carry the intra-bronchial device in a storage configuration, and to load the intra-bronchial device in a delivery configuration into the sheath, and the intra-bronchial device carried in the supply tool in a storage configuration. The delivery catheter may include a stabilization member arranged to be carried in the delivery catheter and to stabilize the intra-bronchial device at the location during unsheathing. The supply tool may include a storage portion, a delivery portion, and a configuration-changing portion coupled between the storage portion and the delivery portion.

A further embodiment of the invention provides a method of delivering an intra-bronchial device into an air passageway. The method includes the steps of moving the intra-bronchial device from a storage configuration to a delivery configuration, loading the intra-bronchial device in a delivery configuration into a distal end of a deployment lumen, advancing the distal end down a trachea to a location in an air passageway, stabilizing the intra-bronchial device at the location, and retracting the distal end to deploy the intra-bronchial device. The loading step may include the further steps of moving the intra-bronchial device into the distal end from a supply tool associated with the distal end, and disassociating the supply tool from the distal end.

A still further embodiment of the invention provides a method of delivering an intra-bronchial device into an air passageway. The method includes the steps of moving the intra-bronchial device from a storage configuration to a delivery configuration and loading the intra-bronchial device in a delivery configuration into a sheath arranged for deployment of the intra-bronchial device by unsheathing. The method also includes advancing the sheathed intra-bronchial device down a trachea to a location in an air passageway, and stabilizing the intra-bronchial device at the location while unsheathing the intra-bronchial device.

Yet a still further embodiment of the invention provides an assembly for delivering an intra-bronchial device into an air passageway. The assembly includes means for advancing the intra-bronchial device in a sheathed delivery configuration down a trachea to a location in an air passageway and for delivering the intra-bronchial device by unsheathing, means for stabilizing the intra-bronchial device at the location while unsheathing the intra-bronchial device, and means associated with the sheath means for carrying the intra-bronchial device in a storage configuration, and for loading the intra-bronchial device in a delivery configuration into the sheath means.

Another embodiment of the invention provides a delivery catheter for delivering an intra-bronchial device into an air passageway. The delivery catheter includes a deployment catheter having a deployment lumen with a distal end arranged to receive the intra-bronchial device in a delivery configuration, the distal end being further arranged to be passed down a trachea and advanced into an air passageway to a location for deployment of the intra-bronchial device. The delivery catheter also includes a stabilization member arranged to be carried in the deployment lumen and to stabilize the intra-bronchial device at the location during retraction of the distal end, the deployment catheter and the stabilization member being longitudinally moveable relative to each other such that retracting the deployment catheter deploys the intra-bronchial device. The delivery catheter may include an intra-bronchial device carried in the distal end in a delivery configuration. The delivery catheter may include an actuator arranged to allow an operator to maintain the stabilization member in a position while retracting the deployment catheter. The delivery catheter may be sterilizable.

A further embodiment of the invention provides a method of delivering an intra-bronchial device into an air passageway. The method includes the steps of advancing a sheathed intra-bronchial device down a trachea to a location in an air passageway for deployment, stabilizing the intra-bronchial device at the location, and retracting the sheath to deploy the stabilized intra-bronchial device. The method may include the further step of loading the intra-bronchial device in a delivery configuration into a sheath.

A still further embodiment of the invention provides a delivery catheter for delivering an intra-bronchial device into an air passageway. The delivery catheter includes a strain-relief catheter having a lumen, and a deployment catheter at least partially carried within the strain-relief catheter lumen and having a deployment lumen with a distal end arranged to receive the intra-bronchial device in a delivery configuration, the distal end being further arranged to be passed down a trachea and advanced into an air passageway to a location for deployment of the intra-bronchial device. The delivery catheter further includes a stabilization member arranged to be carried with the deployment lumen and to stabilize the received intra-bronchial device at the location during retraction of the distal end, the stabilization member and the strain-relief catheter being coupled such that one is not longitudinally moveable relative to the other, and the deployment catheter and the stabilization member being longitudinally moveable relative to each other such that retracting the deployment catheter deploys the intra-bronchial device. The delivery catheter may include an intra-bronchial device in a delivery configuration carried in the distal end. The delivery catheter may include an actuator having a first portion coupling the stabilization member and the strain-relief catheter and allowing an operator to maintain the stabilization member in a position, and a second portion allowing the operator to retract the deployment catheter.

Another embodiment of the invention provides a delivery catheter for delivering an intra-bronchial device into an air passageway. The assembly including a delivery catheter having a distal sheath arranged to receive the intra-bronchial device in a delivery configuration and to deploy the intra-bronchial device by unsheathing, the distal sheath being further arranged to be passed down a trachea and advanced to a location in an air passageway for deployment of the intra-bronchial device, and a stabilization member arranged to be carried in the delivery catheter and to stabilize the intra-bronchial device at the location during unsheathing. The delivery catheter may further include the intra-bronchial device carried in the distal sheath in a delivery configuration.

Yet another embodiment of the invention provides a delivery catheter for delivering an intra-bronchial device into an air passageway. The delivery catheter includes sheathing means for receiving the intra-bronchial device in a delivery configuration and for delivering the intra-bronchial device by unsheathing, means for advancing the sheathed intra-bronchial device down a trachea to a location in an air passageway for deployment. The delivery catheter also includes means carried within the advancing means for stabilizing the intra-bronchial device at the location while unsheathing the intra-bronchial device, the sheathing means and the stabilization means being longitudinally moveable relative to each other such that retracting the sheathing means unsheathes the intra-bronchial device at the location. The delivery catheter may further include an intra-bronchial device in a delivery configuration carried in the sheathing means.

An embodiment of the invention provides an intra-bronchial device supply tool for supplying an intra-bronchial device in a delivery configuration to a deployment lumen. The supply tool including a storage portion arranged for carrying the intra-bronchial device in a storage configuration, and a delivery portion for loading the intra-bronchial device in a delivery configuration into a deployment lumen, and having an end configured for association with the deployment lumen. The supply tool also includes a configuration-changing portion coupled between the storage portion and the delivery portion, and arranged in cooperation with a pushing member for changing the intra-bronchial device from the storage configuration to the delivery configuration. The storage portion may be further arranged for carrying a plurality of sizes of intra-bronchial devices. The pushing member may be further arranged to move the intra-bronchial device a predetermined distance into the deployment lumen. The supply tool may be further arranged to allow sterilization of the intra-bronchial device carried therein, and the sterilization may includes ethylene oxide (EtO). The supply tool may further include the intra-bronchial device in a storage configuration carried in the storage portion.

A further embodiment of the invention provides an intra-bronchial device supply tool for supplying an intra-bronchial device in a delivery configuration to a deployment lumen. The tool includes a storage portion arranged for carrying the intra-bronchial device in a storage configuration, a delivery portion arranged for loading the intra-bronchial device in a delivery configuration into the deployment lumen and having an end configured for association with the deployment lumen. The tool also includes a longitudinal configuration-changing portion coupled between the storage portion and the delivery portion, and arranged for changing the intra-bronchial device from the storage configuration to the delivery configuration as the intra-bronchial device is moved from the storage portion to the delivery portion, and a pushing member arranged for moving the intra-bronchial device from the storage portion to the delivery portion. The configuration-changing portion may include a narrowing passage between the storage portion and the delivery portion. The configuration-changing portion may include a passage having a continuously varying diameter. The configuration-changing portion may include a passage that, as the intra-bronchial device moves from the storage portion to the delivery portion, presents the intra-bronchial device with a continuously reducing diameter. The supply tool may further include a coupler that associates the delivery portion and the deployment lumen for loading the intra-bronchial device. The pushing member may be further arranged to move the intra-bronchial device a predetermined distance into the deployment lumen.

Another embodiment includes a method of supplying an intra-bronchial device in a delivery configuration to a deployment lumen. The method includes the steps of carrying the intra-bronchial device in a storage configuration, changing the intra-bronchial device from the storage configuration to a delivery configuration, and loading the intra-bronchial device in a delivery configuration into the deployment lumen. The changing step may include the further step of moving the intra-bronchial device through a narrowing passage. The loading step may include the further step of moving the intra-bronchial device a predetermined distance into the deployment lumen.

An embodiment of the invention provides an intra-bronchial device supply tool for supplying an intra-bronchial device in a delivery configuration to a deployment lumen. The tool includes storage means for carrying the intra-bronchial device in a storage configuration, delivery means for loading the intra-bronchial device in a delivery configuration into the deployment lumen, configuration changing means coupled between the storage means and the delivery means for changing the intra-bronchial device from the storage configuration to the delivery configuration as the intra-bronchial device is moved from the storage means to the delivery means, and moving means for moving the intra-bronchial device from the storage means to the delivery means.

A still further embodiment of the invention provides a kit for delivering an intra-bronchial device into an air passageway. The kit includes a delivery catheter having a deployment lumen with a distal end arranged to receive the intra-bronchial device in a delivery configuration, and arranged to be passed down a trachea and advanced to a location in an air passageway for deployment of the intra-bronchial device, and to deploy the intra-bronchial device by retracting the deployment catheter. The kit further includes a plurality of bronchial device supply tools, each arranged to carry one intra-bronchial device in a storage configuration, and one intra-bronchial device in a storage configuration carried to each supply tool, each supply tool carrying a different intra-bronchial device.

### BRIEF DESCRIPTION OF THE DRAWINGS

The features of the present invention which are believed to be novel are set forth with particularity in the appended claims. The invention, together with further objects and advantages thereof, may best be understood by making reference to the following description taken in conjunction with the accompanying drawings, in the several figures of which like referenced numerals identify like elements, and wherein:

FIG. 1 is a sectional view of a respiratory system;

FIG. 2 illustrates an assembly for delivering an intra-bronchial device into an air passageway including a delivery catheter, according to an embodiment of the invention;

FIG. 3 illustrates a cross-sectional view of the delivery catheter of FIG. 2, according to an embodiment of the invention;

FIG. 4 illustrates a longitudinal-sectional view of the distal portion of the delivery catheter in a configuration to receive an intra-bronchial device, according to an embodiment of the invention;

FIG. 5 illustrates a longitudinal-section view of the intra-bronchial device supply tool removably associated with the distal tip of the deployment catheter, according to an embodiment of the invention;

FIG. 6 is a longitudinal-sectional view of the supply tool of FIG. 5 illustrating the configuration of the intra-bronchial device being changed from a storage configuration to a delivery configuration, according to an embodiment of the invention;

FIG. 7 is a longitudinal-sectional view of the supply tool of FIG. 5 illustrating the configuration of the intra-bronchial device having been changed from a storage configuration to a delivery configuration, according to an embodiment of the invention;

FIG. 8 is a longitudinal-sectional view of the supply tool of FIG. 5 illustrating the configuration of the intra-bronchial device after having been loaded into the distal end of the deployment catheter, according to an embodiment of the invention;

FIG. 9 is a longitudinal-sectional view of the supply tool of FIG. 5 illustrating the distal end of the deployment catheter having been disassociated from the supply tool, according to an embodiment of the invention;

FIG. 10 illustrates a bronchoscope transorally placed into the trachea of a patient, and the delivery catheter carried in a working lumen of the bronchoscope, in accordance with an embodiment of the invention;

FIG. 11 illustrates the distal end of the bronchoscope and a partial cross-section view of the distal end of the deployment catheter advanced to a location in an air passageway for deployment of the intra-bronchial device, in accordance with an embodiment of the invention;

FIG. 12 illustrates a starting configuration where the device is carried in a delivery configuration in the distal end and proximate to the stabilization seat, in accordance with an embodiment of the invention;

FIG. 13 illustrates an intermediate step where the stabilization seat stabilizes the device at the location for deployment while the distal end is being moved proximally, in accordance with an embodiment of the invention; and

FIG. 14 illustrates a final step where the distal end has been moved proximally until the device has been unsheathed, in accordance with an embodiment of the invention.

### DETAILED DESCRIPTION

In the following detailed description of exemplary embodiments of the invention, reference is made to the accompanying drawings, which form a part hereof. The detailed description and the drawings illustrate specific exemplary embodiments by which the invention may be practiced. These embodiments are described in sufficient detail to enable those skilled in the art to practice the invention. It is understood that other embodiments may be utilized, and other changes may be made, without departing from the spirit or scope of the present invention. The following detailed description is therefore not to be taken in a limiting sense, and the scope of the present invention is defined by the appended claims.

Throughout the specification and claims, the following terms take the meanings explicitly associated herein unless the context dictates otherwise. The meaning of “a”, “an”, and “the” include plural references. The meaning of “in” includes “in” and “on.” Additionally, a reference to the singular includes a reference to the plural unless otherwise stated or inconsistent with the disclosure herein.

Additionally, throughout the specification, claims, and drawings, the term “proximal” means nearest the trachea or the mouth, and “distal” means nearest the alveoli.

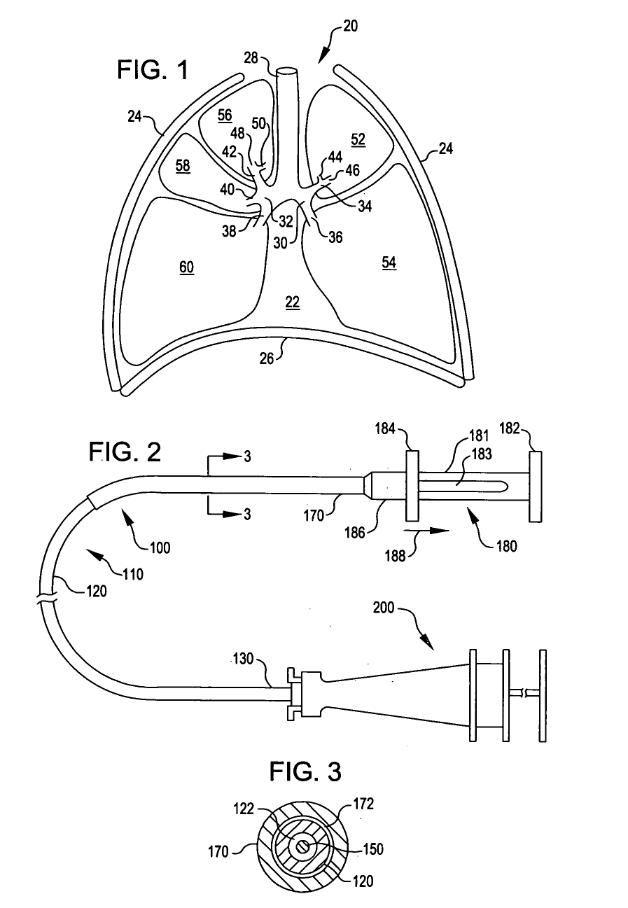

FIG. 1 is a sectional view of a respiratory system. The respiratory system 20 resides within the thorax 22 that occupies a space defined by the chest wall 24 and the diaphragm 26. The respiratory system 20 includes trachea 28; left mainstem bronchus 30 and right mainstem bronchus 32 (primary, or first generation); and lobar bronchial branches 34, 36, 38, 40, and 42 (second generation). FIG. 1 also illustrates segmental branches 44, 46, 48, 49, and 50 (third generation). The respiratory system 20 further includes left lung lobes 52 and 54 and right lung lobes 56, 58, and 60. Each bronchial branch and sub-branch communicates with a different portion of a lung lobe, either an entire lung lobe or a portion thereof. As used herein, the term “air passageway” is meant to denote either a bronchi or bronchioli, and typically means a bronchial branch of any generation.

The air passageways branch out, much like the roots of a tree. The bronchial segments branch into six generations or orders, and the bronchioles branch into approximately another three to eight generations or orders. Typically, each generation has a smaller diameter than does its predecessor. The inside diameter of a generation varies depending on the particular bronchial branch, and further varies between individuals. For example, a typical lobar bronchus 42 (third generation) providing air circulation to the upper right lobe 56 has an internal diameter of approximately 1 cm. A typical segmental bronchi 48 (fourth generation) has an internal diameter of approximately 4 to 7 mm. The fifth and sixth generations (no reference numbers) are each proportionately smaller. The bronchial segments include annular ligaments and irregularly located cartilages that provide structure and resilience. Furthermore, the inside diameters of air passageways is not static. They expand when a person inhales and contract when a person exhales.

Intra-bronchial devices may be delivered and deployed in one or more air passageways in a patient, depending on the therapeutic objective. As described previously, variation in air passageway diameters requires different sizes of intra-bronchial devices be available to correctly fit a device to an air passageway.

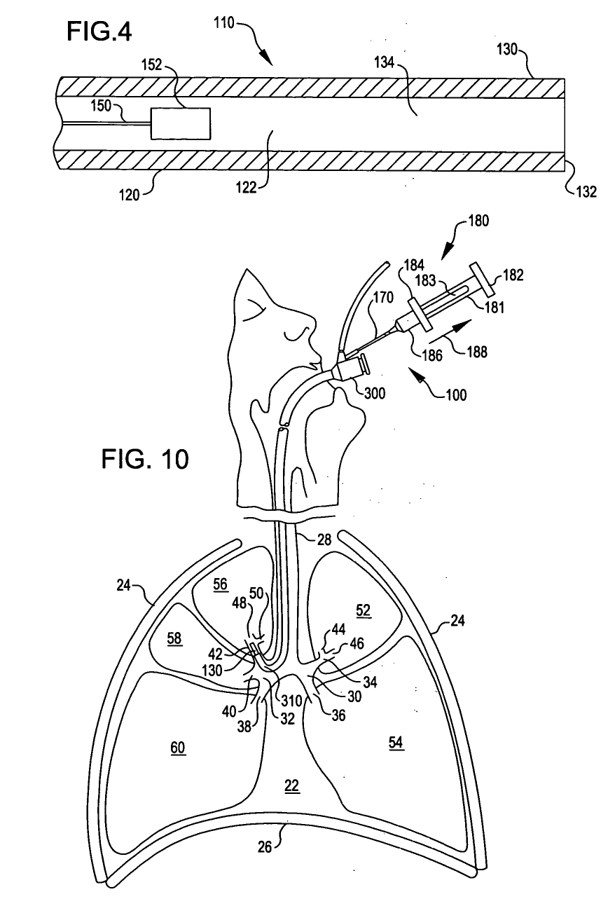

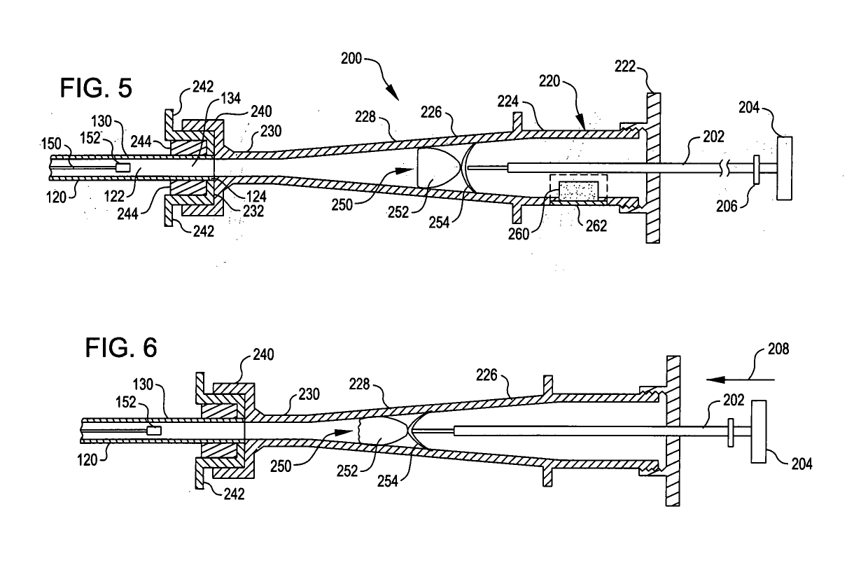

FIGS. 2-5 are related. FIG. 2 illustrates an assembly 100 for delivering an intra-bronchial device in an air passageway including delivery catheter 110, according to an embodiment of the invention. The assembly 100 includes a delivery catheter 110, and a bronchial device supply tool 200. FIG. 3 illustrates a cross-sectional view of the delivery catheter 110 of FIG. 2, according to an embodiment of the invention. FIG. 4 illustrates a longitudinal sectional view of the distal portion of the delivery catheter 110 in a configuration to receive an intra-bronchial device, according to an embodiment of the invention. A description of the intra-bronchial device supply tool 200 is provided in conjunction with FIG. 5.

The delivery catheter 110 includes a deployment catheter 120, a stabilization member 150, an actuator 180, and optimally a strain-relief catheter 170. The delivery catheter 110 is dimensioned for use in a working channel of a bronchoscope or other instrument providing endoscopic viewing and instrumentation in air passageways, and may be sterilizable.

The strain-relief catheter 170 has a lumen 172, and is structured to surround and protect a proximal portion of the deployment catheter 120. The strain-relief catheter 170 is further structured for being griped to maneuver the delivery catheter 110, and to anchor the delivery catheter 110 to a bronchoscope.

The deployment catheter 120 includes a deployment lumen 122 that is structured to be partially carried within lumen 172, and steerable into air passageways. The distal end 130 portion of the deployment lumen 122 is arranged to receive the intra-bronchial device in a delivery configuration, and further arranged for passage down a trachea and advancement into an air passageway to a location for deployment of the intra-bronchial device. The stabilization member 150 is structured to be carried within deployment lumen 122, and to flex in conjunction with steering of the deployment catheter 120.

The actuator 180 is an extracorporeal member for steering the deployment catheter 110, and for retracting the distal end 130 to deploy an intra-bronchial device carried in the deployment catheter 120. The actuator 180 includes a body 181, a fixed handle 182, a movable handle 184, and a fitting portion 186 for anchoring the actuator 180 to a bronchoscope. The actuator 180 may have any configuration and appropriate ergonomics known to those skilled in the art, such as a syringe, gun, pencil, or other design. The actuator 180 is illustrated for convenience in FIG. 2 with a “reverse” syringe configuration for a thumb to rest against the fixed handle 182, and for fingers of the same hand to pull (retract) the moveable handle 184 toward the fixed handle 182 in the retraction direction 188. The body 181 is structured to couple the proximal end of the stabilization member 150 and the proximal end of the strain-relief catheter 170 such that one is not longitudinally moveable relative to the other. The fixed handle 182 is coupled to the body 181. The deployment catheter 120 is structurally coupled to the moveable handle 184, which is arranged to move (retract) in the retraction direction 188 to retract the deployment catheter 120 while the stabilization member 150 and the strain-relief catheter do not move longitudinally. As will be described in conjunction with subsequent figures, movement in the retraction direction 188 will deploy the intra-bronchial device from deployment catheter 120.

The assembly for deploying an intra-bronchial device 110 is typically provided with the bronchial device supply tool 200 removably associated with the distal end 130 of the deployment catheter 120, and an intra-bronchial device in a storage configuration carried in a storage portion of the tool 200. As described in subsequent figures, the supply tool 200 is used to load the intra-bronchial device in a delivery configuration into a distal end 130. The supply tool 200 is then disassociated from the deployment catheter 120, and the distal end 130 of the deployment catheter is ready to be passed through a working lumen of a bronchoscope, and advanced into a location in an air passageway for deployment of the device.

Turning now to FIG. 4, the delivery catheter 110 includes the deployment catheter 120 having a distal end 130 and a distal tip 132, and the stabilization member 150 having an intra-bronchial device stabilization seat 152 on a distal end. The distal portion of the delivery catheter 110 also includes a portion of the deployment lumen 122 between the distal tip 132 and the stabilization seat 152 defining a distal sheath 134.

The deployment lumen 122 includes a material having a relatively hard and/or smooth surface to prevent anchors or other features of an intra-bronchial device from engaging the deployment lumen 122 and interfering with deployment. As described below, the material may be medical-catheter-grade nylon 12. The stabilization seat 152 is arranged to stabilize the received intra-bronchial device in the distal end of the deployment catheter lumen.

FIG. 4 further illustrates a device-receiving configuration. The stabilization seat 152 is located proximal of the distal tip 132 in lumen 122 by a distance preferably of at least equal to a length of a device in a delivery configuration, thus defining a space for receiving the device in a delivery configuration that is wholly within the lumen 122. This space forms the distal sheath 134, which is arranged to receive an intra-bronchial device in a delivery configuration.

An embodiment of the delivery catheter 110 was constructed using sterilizable materials. The strain-relief catheter 170 includes straight-walled, reinforced medial-grade polyimide tubing having an outside diameter of approximately 0.076 inches, selected to be smaller than the 2 mm working lumen of a common bronchoscope. The strain-relief catheter further has a length of approximately 12 inches, and its lumen 172 has an inside diameter of approximately 0.055-0.060 inches. The deployment catheter 120 is made from a tapered, nylon 12 tubing approximately 45 inches long. It has a proximal end outside diameter of approximately 0.050-0.055 inches with a deployment lumen 122 inside diameter of approximately 0.040 inches, and a distal end 130 outside diameter of approximately 0.076 inches, and a deployment lumen 122 inside diameter of approximately 0.065 inches.

The stabilization member 150 is made from a Teflon-coated, stainless steel wire coiled about a center “core” wire approximately 44 inches long (slightly shorter than the deployment catheter), has a diameter of 0.038-0.040 inches, and includes a stabilization seat 152 carried on its distal end. The last, approximately four inches of the distal end of the stabilization member 150 is designed to be more flexible than the remaining portion of the wire. The proximal end of the coiled wire is terminated with solid wire for crimping in place to the actuator 180. The stabilization seat 152 is about 0.100 inches long, has an outside diameter of 0.060 inches, which is slightly less than the diameter of delivery lumen 122, and contains a concave portion in a face arranged to contact a proximal end of the intra-bronchial device.

An embodiment of actuator 180 was constructed utilizing sterilizable plastic. The moveable handle 184 is coupled to the deployment catheter 120, and a slot 183 is used to limit the movement of the moveable handle 184 and correspondingly the movement of the deployment catheter 120. The strain-relief catheter 170 and the stabilization member 150 are coupled to the body 181, with provision for the deployment catheter 120 to move relative to the strain-relief catheter 170 and the stabilization member 150 in the retraction direction 188.

FIG. 5 illustrates a longitudinal-section view of the intra-bronchial device supply tool 200 removably associated with the distal tip 130 of the deployment catheter 120, according to an embodiment of the invention. The supply tool 200 is illustrated with an intra-bronchial device 250 that comprises an obstructing member 252 and anchors 254. This type of intra-bronchial device is described in the anchored device applications previously incorporated herein by reference. The device 250 is carried in a storage configuration. The supply tool 200 includes a pushing member 202, a body 220, and a retaining clip 242.

The body 220 includes a cap 222, a housing 224 and an associating portion 240. The cap 222 is arranged for coupling with the housing 224 in any manner known in the art compatible with the materials and intended function of the supply tool 200, which may include threads or adhesive. The cap 222 includes an aperture, and optionally, a support for the pushing member 202. Pushing member 202 includes a plunger 204 and a stop 206. The housing 224 includes a storage portion 226, a configuration-changing portion 228, a delivery portion 230, and an associating portion 240. The associating portion 240 includes a mating face 232, a retaining clip 242, and a gasket 244.

In an embodiment, the supply tool 200 is a non-reusable product arranged to store, transport, and load a single device. In this embodiment, the supply tool 200 is made from any plastic material known to those in the art compatible with its intended function. The housing 224 is generally cylindrical, and has a generally conical-shaped barrel forming the storage portion 226, the configuration-changing portion 228, and the delivery portion 230. The narrowing or conical shape of the storage portion 226 allows it to store a plurality of sizes of an intra-bronchial device 250 by positioning a particular device to be stored at a diameter appropriate for storage of that size. The narrowing or conical shape of the configuration-changing portion 228 compresses or collapses an intra-bronchial device 250 into a delivery configuration as it is moved from the storage portion 226 to the delivery portion 230.

While FIG. 5 illustrates both the storage portion 226 and configuration-changing portion 228 as including a narrowing passage with a uniformly decreasing diameter, each portion may have unique characteristics. For example, the storage portion 226 may comprise a series of discrete portions becoming narrower proceeding from the cap 222 end of the body 220 toward the associating portion 240, each discrete portion being dimensioned to store a size of the intra-bronchial device 250. Furthermore, the configuration-changing portion 228 may have a continuously changing diameter that changes less rapidly toward the delivery portion 230.

The delivery portion 230 is intended to establish the delivery configuration for the intra-bronchial device 250, and to match the diameter of the deployment lumen 122 for loading the device 250 into the deployment lumen 122. FIG. 5 illustrates the delivery portion 230 having a longitudinal dimension approximating a length of the intra-bronchial device 250 in the delivery configuration. However, this longitudinal dimension may be reduced such that the delivery portion 230 is the smallest diameter of the configuration-changing portion 228.

Associating portion 240 is structured to removably associate the supply tool 200 with the distal end 130 of deployment catheter 120. The mating face 232 is generally perpendicular to a longitudinal axis of the supply tool 200, and arranged to mate with the distal tip 132 such that the longitudinal axis of the deployment lumen 122 and the delivery portion 230 align for loading the intra-bronchial device 250 in a delivery configuration into the deployment lumen 122. The retaining clip 242 and the gasket 244 are arranged to further control the alignment of the delivery portion 230 and the deployment lumen 122, and to removably associate or couple the supply tool 200 to the deployment catheter 120.

An embodiment of the body 220 may include a breathable, sterile barrier 262 carried in breathable barrier opening 260. The breathable, sterile barrier 262 may be any breathable barrier known in the art for facilitating in-package sterilization, such as TYVEK® brand substrate, a registered trademark of du Pont de Nemours and Company. The breathable barrier 262 allows the device 250 to be sterilized after it is stored in the storage part 226. Sterilization methods may include any method known to those skilled in the art, including using an ethylene oxide (EtO) gas.

The cap 222 is arranged in cooperation with the housing 224 to form an enclosed space for storing and protecting the intra-bronchial device 250. The cap 222 may also be arranged in cooperation with the housing 224 to form a gripable structure for operation of the pushing member 202. The pushing member 202 may be made from any material known in the art suitable for the intended purpose of the tool 200, including a metal or a plastic. One end of the pushing member 202 is arranged for placement and pushing on the device 250. The other end includes a plunger 204, and a stop 206 arranged to limit the advancement of the pushing member 220.

In use, a single intra-bronchial device 250 is placed proximate to the storage portion 226, and the cap 222 with the pushing member 202 is coupled to the housing 224. The plunger 204 is moved in direction 208 to push on and move the device 250 toward the storage portion 226. This moves the intra-bronchial device 250 into an increasingly narrow portion of the storage portion 226 until it assumes a storage configuration in storage portion 226. While the storage configuration may be different for different types of devices, device 250 typically would be advanced until a portion of its obstructing member 252 and its anchors 254 loosely contact the walls of the storage portion, thus holding the device 250 in a position. If not previously associated, the distal end 130 is associated with the supply tool 200 with the retaining clip 242, forming the assembly 100 described in FIG. 2. The mating face 232 is brought proximate to, and aligned with, the distal tip 132, and the retaining clip 242 operated to complete the association. If sterilization is to be performed, the device 250 may be sterilized through the breathable, sterile barrier 262 using an ethylene oxide (EtO) gas.

FIG. 6 is a longitudinal-sectional view of the supply tool 200 of FIG. 5 illustrating the configuration of intra-bronchial device 250 being changed from a storage configuration to a delivery configuration, according to an embodiment of the invention. Some reference numbers are omitted for clarity. When the device 250 is needed for delivery into an air passageway, the pushing member 202 is advanced as illustrated in FIG. 6 in the direction indicated by arrow 208 toward the distal end 130. This advancement moves the device 250 into increasingly narrower longitudinal segments of the configuration-changing portion 228 of the housing 224, correspondingly reducing radial dimensions of the device 250 toward its delivery configuration.

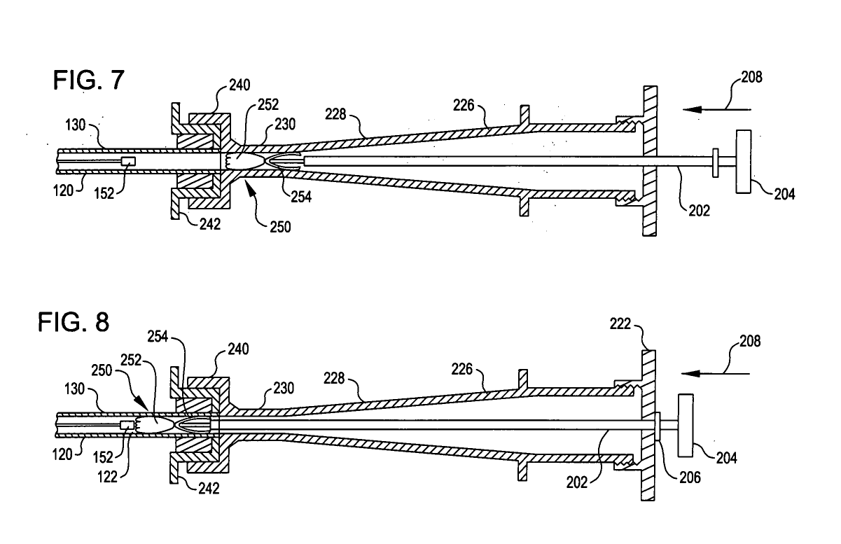

FIG. 7 is a longitudinal-sectional view of the supply tool 200 of FIG. 5 illustrating the configuration of intra-bronchial device 250 having been changed from a storage configuration to a delivery configuration, according to an embodiment of the invention. The pushing member 202 has been further advanced in the direction indicated by arrow 208 toward the distal end 130. This further advancement has moved the device 250 through increasingly narrower longitudinal segments of the configuration-changing portion 228 and into the delivery portion 230 of the housing 224, correspondingly reducing radial dimensions of the device 250 to its delivery configuration. As described above, the longitudinal dimension of the delivery portion 230 may be reduced such that the delivery portion 230 is at the smallest diameter of the configuration-changing portion 228.

FIG. 8 is a longitudinal-sectional view of the supply tool 200 of FIG. 5 illustrating the configuration of intra-bronchial device 250 having been loaded into the distal end 130 of the deployment catheter 120, according to an embodiment of the invention. The pushing member 202 has been further advanced in the direction indicated by arrow 208, moving the device 250, in the delivery configuration, into the delivery lumen 122 proximate to the stabilization seat 152 in preparation for deployment. Advancement of the pushing member 202 may be limited by the stop 206 moving proximate to the cap 222. The stop 222 may be located on pushing member 202 to limit the advancement of the device 250 into the delivery lumen 122 to a predetermined distance.

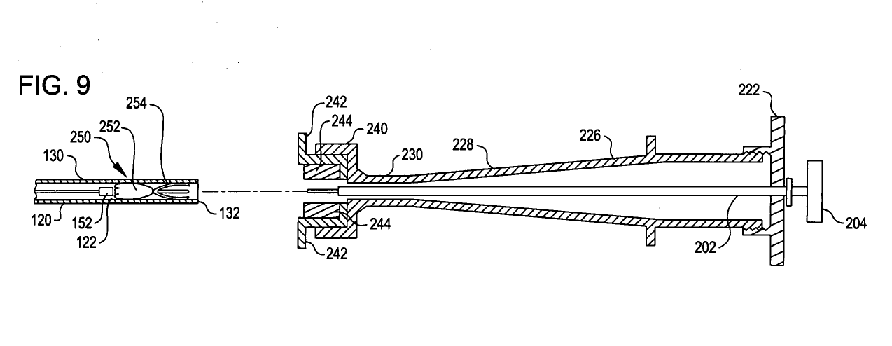

FIG. 9 is a longitudinal-sectional view of the supply tool 200 of FIG. 5 illustrating the distal end 130 of the deployment catheter 120 having been disassociated from the supply tool 200, according to an embodiment of the invention. The retainer clips 242 are released, and the distal end 130 and the association portion 240 of the tool 200 can be moved apart to disassociate. With disassociation, the distal end 130 is ready for advancement into an air passageway and deployment of the device 250.

If a particular type of intra-bronchial device can be stored in a delivery configuration, the supply tool 200 can be used to preload the device 250 into the distal end 130. This allows the assembly 100 of FIG. 2 to be provided without the supply tool 200.

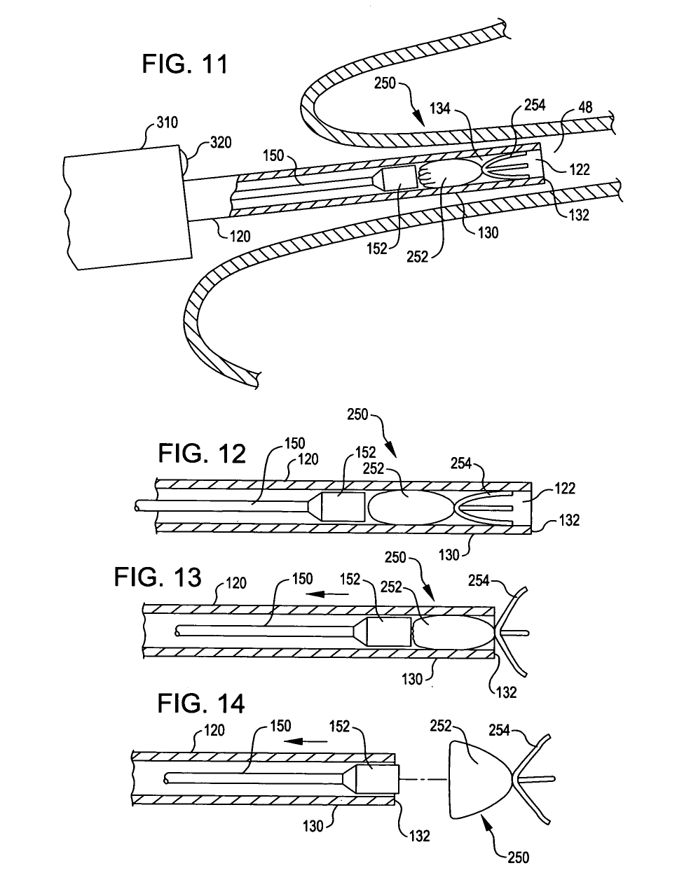

A final series of figures illustrate the delivery catheter 110 delivering the intra-bronchial device 250 into an air passageway. FIG. 10 illustrates a bronchoscope 300 transorally placed in the trachea 28 of a patient, and the delivery catheter 110 carried in a working lumen of the bronchoscope, in accordance with an embodiment of the invention. FIG. 11 illustrates the distal end 310 of the bronchoscope 300 and a partial longitudinal-section view of the distal end 130 of the deployment catheter 120 advanced to a location in an air passageway for deployment of the intra-bronchial device 250, in accordance with an embodiment of the invention.

An initial step in delivering an intra-bronchial device 250 requires that the distal end 130 of the deployment catheter 120, also referred to as distal sheath 134, receive the device selected for delivery. An aspect of the invention provides alternative ways for loading a selected intra-bronchial device into the distal end 130. An assembly 100 may be selected having an appropriately sized intra-bronchial device 250 carried in loading tool 200. The loading tool 200 is used to move the intra-bronchial device 250 into a delivery configuration and load it into the distal tip 130, and then disassociated, as described in conjunction with FIGS. 6-9. In another embodiment, a plurality of loading tools 200 is available, each containing a different size of intra-bronchial device 250. A loading tool 200 may be selected having an appropriately sized device 250 carried therein. The selected loading tool 200 is associated with the distal tip 130 of the deployment catheter 120 and used to load the device 250 as described above. In a further embodiment where the deployment catheter 120 is provided with the intra-bronchial device 250 preloaded in the distal tip 130, a deployment catheter 120 carrying an appropriately sized device 250 is selected.

Bronchoscope 300 is transorally placed in the trachea 28 using known techniques, and its distal end 310 is advanced and steered into a selected air passageway near a location for deployment of the intra-bronchial device 250. For the purposes describing an embodiment of the invention, segmental branch 48 is used to illustrate a location in an air passageway for deployment. The distal end 310 may be steered using the visualization element 320, or other visualization technique known to those in the art. Once the distal end 310 is within working distance of the location, the distal end 130 of the deployment catheter 120 carrying a selected intra-bronchial device 250 is advanced from the distal end of a working lumen of the bronchoscope 300 to the location as illustrated in FIG. 11.

FIGS. 12-14 illustrate partial longitudinal-section views of the distal end 130 of the deployment catheter 120 advanced to the location in an air passageway for deployment of the intra-bronchial device 250 and deploying the device 250, in accordance with an embodiment of the invention. FIG. 12 illustrates a starting configuration where the device 250 is carried in a delivery configuration in the distal end 130 and proximate to the stabilization seat 152. Once the distal end 130 is positioned at the location, the moveable handle 184 of the actuator 180 is moved in the retraction direction 188 as illustrated in FIG. 10. Since moveable handle 184 is coupled to the deployment catheter 120 and the stabilization member 150 is coupled to the fixed body 181, movement of the moveable handle 184 in the retraction direction 188 moves the distal end 130 proximal of the device 250 while the stabilization seat 152 stabilizes the device 250 at the location.

FIG. 13 illustrates an intermediate step where the stabilization seat 152 stabilizes the device 250 at the location for deployment while the distal end 130 is being moved proximally. In the illustrated intermediate step, the anchors 254 have deployed while the obstructing member 254 remains within the delivery lumen 122. Continuing movement of the handle 184 in the retraction direction 188 further moves the distal end 130 proximal of the device 250 and completes deployment of the device 250 at the location. FIG. 14 illustrates a final step where the distal end 130 has been moved proximally until the device 250 has been unsheathed and deployed at the location for deployment.

In a further step, the deployment catheter 120 is removed from the bronchoscope 300. In another embodiment, the deployment catheter 120 can be removed and reloaded with another selected device 250 from another loading tool 200, and the steps repeated to deploy the another device 250 at another location. In a further embodiment, a deployment catheter 120 with another preloaded device 250 can be selected and the steps repeated to deploy the another device 250 at another location.

The invention disclosed herein is not limited to use with the particular method illustrated herein. For example, the delivery catheter 110 may be used alone to perform the delivery, may be extended from the bronchoscope 300, or used in conjunction with the bronchoscope 300. For purposes of this description, the delivery has been described with reference to only the catheter 110 being extended from the bronchoscope 300.

Although the present invention has been described in considerable detail with reference to certain preferred embodiments, other embodiments are possible. Therefore, the spirit or scope of the appended claims should not be limited to the description of the embodiments contained herein. It is intended that the invention resides in the claims hereinafter appended.

What is claimed is:

1. An assembly for delivering an intra-bronchial device into an air passageway, the assembly comprising:
a delivery catheter having a deployment lumen with a distal end arranged to receive the intra-bronchial device in a delivery configuration, and arranged to be passed down a trachea and advanced to a location in an air passageway for deployment of the intra-bronchial device, and to deploy the intra-bronchial device by retracting the deployment catheter;
a bronchial device supply tool associated with the distal end, and arranged to carry the intra-bronchial device in a storage configuration and to load the intra-bronchial device in a delivery configuration into the deployment lumen of distal end; and
the intra-bronchial device carried in the supply tool in a storage configuration.

2. The assembly of claim 1, wherein the delivery catheter includes a deployment catheter having the deployment lumen, and a stabilization member arranged to be carried in the deployment lumen and to stabilize the intra-bronchial device at the location during retraction of the distal end.

3. The assembly of claim 1, wherein the supply tool includes a storage portion, a delivery portion, and a configuration-changing portion coupled between the storage portion and the delivery portion.

4. An assembly for delivering an intra-bronchial device into an air passageway, the assembly comprising:
a delivery catheter having a distal sheath arranged to receive the intra-bronchial device in a delivery configuration and to deploy the intra-bronchial device by unsheathing, the distal sheath being further arranged to be passed down a trachea and advanced to a location in an air passageway for deployment of the intra-bronchial device;
a bronchial device supply tool removably coupled to the distal end and arranged to carry the intra-bronchial device in a storage configuration, and to load the intra-bronchial device in a delivery configuration into the sheath; and
the intra-bronchial device carried in the supply tool in a storage configuration.

5. The assembly of claim 4, wherein the delivery catheter includes a stabilization member arranged to be carried in the delivery catheter and to stabilize the intra-bronchial device at the location during unsheathing.

6. The assembly of claim 4, wherein the supply tool includes a storage portion, a delivery portion, and a configuration-changing portion coupled between the storage portion and the delivery portion.

7. A method of delivering an intra-bronchial device into an air passageway, comprising the steps of:
moving the intra-bronchial device from a storage configuration to a delivery configuration;
loading the intra-bronchial device in a delivery configuration into a distal end of a deployment lumen;
advancing the distal end down a trachea to a location in an air passageway;
stabilizing the intra-bronchial device at the location; and
retracting the distal end to deploy the intra-bronchial device.

8. The method of claim 7, wherein the loading step includes the further steps of moving the intra-bronchial device into the distal end from a supply tool associated with the distal end, and disassociating the supply tool from the distal end.

9. A method of delivering an intra-bronchial device into an air passageway, comprising the steps of:
moving the intra-bronchial device from a storage configuration to a delivery configuration;
loading the intra-bronchial device in a delivery configuration into a sheath arranged for deployment of the intra-bronchial device by unsheathing;
advancing the sheathed intra-bronchial device down a trachea to a location in an air passageway; and
stabilizing the intra-bronchial device at the location while unsheathing the intra-bronchial device.

10. An assembly for delivering an intra-bronchial device into an air passageway, the assembly comprising:
means for advancing the intra-bronchial device in a sheathed delivery configuration down a trachea to a location in an air passageway and for delivering the intra-bronchial device by unsheathing;
means for stabilizing the intra-bronchial device at the location while unsheathing the intra-bronchial device; and
means associated with the sheath means for carrying the intra-bronchial device in a storage configuration, and for loading the intra-bronchial device in a delivery configuration into the sheath means.

11. A delivery catheter for delivering an intra-bronchial device into an air passageway, the delivery catheter comprising:
a deployment catheter having a deployment lumen with a distal end arranged to receive the intra-bronchial device in a delivery configuration, the distal end being further arranged to be passed down a trachea and advanced into an air passageway to a location for deployment of the intra-bronchial device; and
a stabilization member arranged to be carried in the deployment lumen and to stabilize the intra-bronchial device at the location during retraction of the distal end,
the deployment catheter and the stabilization member being longitudinally moveable relative to each other such that retracting the deployment catheter deploys the intra-bronchial device.

12. The delivery catheter of claim 11, further including an intra-bronchial device carried in the distal end in a delivery configuration.

13. The delivery catheter of claim 11, further including an actuator arranged to allow an operator to maintain the stabilization member in a position while retracting the deployment catheter.

14. The delivery catheter of claim 11, wherein the delivery catheter is sterilizable.

15. A method of delivering an intra-bronchial device into an air passageway, comprising the steps of:
advancing a sheathed intra-bronchial device down a trachea to a location in an air passageway for deployment;
stabilizing the intra-bronchial device at the location; and
retracting the sheath to deploy the stabilized intra-bronchial device.

16. The method of claim 15, including the further step of loading the intra-bronchial device in a delivery configuration into a sheath.

17. A delivery catheter for delivering an intra-bronchial device into an air passageway, the delivery catheter comprising:
a strain-relief catheter having a lumen;
a deployment catheter at least partially carried within the strain-relief catheter lumen and having a deployment lumen with a distal end arranged to receive the intra-bronchial device in a delivery configuration, the distal end being further arranged to be passed down a trachea and advanced into an air passageway to a location for deployment of the intra-bronchial device; and
a stabilization member arranged to be carried with the deployment lumen and to stabilize the received intra-bronchial device at the location during retraction of the distal end,
the stabilization member and the strain-relief catheter being coupled such that one is not longitudinally moveable relative to the other, and the deployment catheter and the stabilization member being longitudinally moveable relative to each other such that retracting the deployment catheter deploys the intra-bronchial device.

18. The delivery catheter of claim 17, further including an intra-bronchial device in a delivery configuration carried in the distal end.

19. The delivery catheter of claim 17, further including an actuator having a first portion coupling the stabilization member and the strain-relief catheter and allowing an operator to maintain the stabilization member in a position, and a second portion allowing the operator to retract the deployment catheter.

20. A delivery catheter for delivering an intra-bronchial device into an air passageway, the assembly comprising:
a delivery catheter having a distal sheath arranged to receive the intra-bronchial device in a delivery configuration and to deploy the intra-bronchial device by unsheathing, the distal sheath being further arranged to be passed down a trachea and advanced to a location in an air passageway for deployment of the intra-bronchial device; and
a stabilization member arranged to be carried in the delivery catheter and to stabilize the intra-bronchial device at the location during unsheathing.

21. The delivery catheter of claim 20, further including the intra-bronchial device carried in the distal sheath in a delivery configuration.

22. A delivery catheter for delivering an intra-bronchial device into an air passageway, the delivery catheter comprising:
sheathing means for receiving the intra-bronchial device in a delivery configuration and for delivering the intra-bronchial device by unsheathing;
means for advancing the sheathed intra-bronchial device down a trachea to a location in an air passageway for deployment; and
means carried within the advancing means for stabilizing the intra-bronchial device at the location while unsheathing the intra-bronchial device,
the sheathing means and the stabilization means being longitudinally moveable relative to each other such that retracting the sheathing means unsheathes the intra-bronchial device at the location.

23. The delivery catheter of claim 22, further including an intra-bronchial device in a delivery configuration carried in the sheathing means.

24. An intra-bronchial device supply tool for supplying an intra-bronchial device in a delivery configuration to a deployment lumen, the tool comprising:
a storage portion arranged for carrying the intra-bronchial device in a storage configuration;
a delivery portion for loading the intra-bronchial device in a delivery configuration into a deployment lumen, and having an end configured for association with the deployment lumen; and
a configuration-changing portion coupled between the storage portion and the delivery portion, and arranged in cooperation with a pushing member for changing the intra-bronchial device from the storage configuration to the delivery configuration.

25. The supply tool of claim 24, wherein the storage portion is further arranged for carrying a plurality of sizes of intra-bronchial devices.

26. The supply tool of claim 24, wherein the pushing member is further arranged to move the intra-bronchial device a predetermined distance into the deployment lumen.

27. The supply tool of claim 24, wherein the supply tool is further arranged to allow sterilization of the intra-bronchial device carried therein.

28. The supply tool of claim 27, wherein the sterilization includes ethylene oxide (EtO).

29. The supply tool of claim 24, further including the intra-bronchial device in a storage configuration carried in the storage portion.

30. An intra-bronchial device supply tool for supplying an intra-bronchial device in a delivery configuration to a deployment lumen, the tool comprising:
a storage portion arranged for carrying the intra-bronchial device in a storage configuration;
a delivery portion arranged for loading the intra-bronchial device in a delivery configuration into the deployment lumen and having an end configured for association with the deployment lumen;
a longitudinal configuration-changing portion coupled between the storage portion and the delivery portion, and arranged for changing the intra-bronchial device from the storage configuration to the delivery configuration as the intra-bronchial device is moved from the storage portion to the delivery portion; and
a pushing member arranged for moving the intra-bronchial device from the storage portion to the delivery portion.

31. The supply tool of claim 30, wherein the configuration-changing portion includes a narrowing passage between the storage portion and the delivery portion.

32. The supply tool of claim 30, wherein the configuration-changing portion includes a passage having a continuously varying diameter.

33. The supply tool of claim 30, wherein the configuration-changing portion includes a passage that, as the intra-bronchial device moves from the storage portion to the delivery portion, presents the intra-bronchial device with a continuously reducing diameter.

34. The supply tool of claim 30, further including a coupler that associates the delivery portion and the deployment lumen for loading the intra-bronchial device.

35. The supply tool of claim 30, wherein the pushing member is further arranged to move the intra-bronchial device a predetermined distance into the deployment lumen.

36. A method of supplying an intra-bronchial device in a delivery configuration to a deployment lumen, the method comprising the steps of:
carrying the intra-bronchial device in a storage configuration;
changing the intra-bronchial device from the storage configuration to a delivery configuration; and
loading the intra-bronchial device in a delivery configuration into the deployment lumen.

37. The method of claim 36, wherein the changing step includes the further step of moving the intra-bronchial device through a narrowing passage.

38. The method of claim 36, wherein the loading step includes the further step of moving the intra-bronchial device a predetermined distance into the deployment lumen.

39. An intra-bronchial device supply tool for supplying an intra-bronchial device in a delivery configuration to a deployment lumen, the tool comprising:
storage means for carrying the intra-bronchial device in a storage configuration;
delivery means for loading the intra-bronchial device in a delivery configuration into the deployment lumen;
configuration changing means coupled between the storage means and the delivery means for changing the intra-bronchial device from the storage configuration to the delivery configuration as the intra-bronchial device is moved from the storage means to the delivery means; and
moving means for moving the intra-bronchial device from the storage means to the delivery means.

40. A kit for delivering an intra-bronchial device into an air passageway, the kit comprising:
a delivery catheter having a deployment lumen with a distal end arranged to receive the intra-bronchial device in a delivery configuration, and arranged to be passed down a trachea and advanced to a location in an air passageway for deployment of the intra-bronchial device, and to deploy the intra-bronchial device by retracting the deployment catheter;
a plurality of bronchial device supply tools, each arranged to carry one intra-bronchial device in a storage configuration; and
one intra-bronchial device in a storage configuration carried with each supply tool, each supply tool carrying a different intra-bronchial device.
